SEQUENCE LISTING (19 sequences)

SEQ ID NO: 1 - DNA, 71 bp, synthetic construct
  source 1..71: mol_type = other DNA
atgcggatcccgcgcatttaaagcgcttcgatgcgtatgggtttgtccgattagctgcgcgaagcttgcgc

SEQ ID NO: 2 - DNA, 71 bp, synthetic construct
  source 1..71: mol_type = other DNA
atgcggatcccgcgcatattaaaggggaggctccctatgggtttgtccgattagctgcgcgaagcttgcgc

SEQ ID NO: 3 - DNA, 71 bp, synthetic construct
  source 1..71: mol_type = other DNA
atgcggatcccgcgcatttaaagcggttcgatgcgtatgggtttgtccgattcggtgcgcgaagcttgcgc

SEQ ID NO: 4 - DNA, 69 bp, synthetic construct
  source 1..69: mol_type = other DNA
atgcggatcccgcgcatttaaagcgtcgatgcgtatgggtttgtccgattagctgcgcgaagcttgcgc

SEQ ID NO: 5 - DNA, 66 bp, synthetic construct
  source 1..66: mol_type = other DNA
atgcggatcccgcgcatttaaagggagacctatgggtttgtccgattagctgcgcgaagcttgcgc

SEQ ID NO: 6 - DNA, 71 bp, synthetic construct; identical to SEQ ID NO: 1
  source 1..71: mol_type = other DNA

SEQ ID NO: 7 - DNA, 71 bp, synthetic construct; identical to SEQ ID NO: 1
  source 1..71: mol_type = other DNA

SEQ ID NO: 8 - DNA, 70 bp, synthetic construct
  source 1..70: mol_type = other DNA
atgcggatcccgcgcatttaaagggggcgctccgtatgggtttgtccgattagctgcgcgaagcttgcgc

SEQ ID NO: 9 - DNA, 79 bp, synthetic construct
  source 1..79: mol_type = other DNA
atgcggatcccgcgcccgacgtcgcatgctcccggccgccatggcggccgcgggaattcgattagcgcgaagcttgcgc

SEQ ID NO: 10 - DNA, 40 bp, synthetic construct
  source 1..40: mol_type = other DNA
atttaaagcggttcgatgcgtatggtttgtccgattcggt

SEQ ID NO: 11 - DNA, 35 bp, synthetic construct
  source 1..35: mol_type = other DNA
atttaaagcggttcgatgcgtatggtttgtccgat

SEQ ID NO: 12 - DNA, 29 bp, synthetic construct
  source 1..29: mol_type = other DNA
agcggttcgatgcgtatggtttgtccgat

SEQ ID NO: 13 - DNA, 36 bp, synthetic construct
  source 1..36: mol_type = other DNA
atttaaagcgcttcgatgcgtatgggtttgtccgat

SEQ ID NO: 14 - DNA, 40 bp, synthetic construct
  source 1..40: mol_type = other DNA
atttaaagggggcgctccgtatgggtttgtccgattagct

SEQ ID NO: 15 - protein, 22 aa, synthetic construct
MRIPRISGSMRMGLSDSVREAC

SEQ ID NO: 16 - protein, 21 aa, synthetic construct
MRIPRISASMRMGLSDLREAC

SEQ ID NO: 17 - protein, 22 aa, synthetic construct
MRIPRIRGRSVWVCPISCAKLA

SEQ ID NO: 18 - DNA, 15 bp, synthetic construct
  source 1..15: mol_type = other DNA
atgcggatcccgcgc

SEQ ID NO: 19 - DNA, 15 bp, synthetic construct
  source 1..15: mol_type = other DNA
gcgcgaagcttgcgc

(19) United States
(12) Patent Application Publication
(10) Pub. No.: US 2026/0235601 A1
(43) Pub. Date: Aug. 13, 2026

(54) APTAMER BASED DETECTION OF SARS-COV-2 INFECTION

(71) Applicant: COUNCIL OF SCIENTIFIC & INDUSTRIAL RESEARCH, New Delhi (IN)

(72) Inventors: Amogh Milind AUTI, Pune (IN); Yogesh Laxmirajam BHANDARI, Pune (IN); Sanjana Sudhir VARMA, Pune (IN); Bhushan Pradosh CHAUDHARI, Pune (IN); Koteswara Rao VAMKUDOTH, Pune (IN)

(21) Appl. No.: 19/152,697

(22) PCT Filed: Jan. 30, 2024
(86) PCT No.: PCT/IN2024/050085

§ 371 Date: Jul. 31, 2025

(30) Foreign Application Priority Data
Jan. 31, 2023 (JP) 202311006157

(51) Int. Cl. G01N 33/569; C12N 1/20; C12N 15/115; C12N 15/70; C12R 1/19; G01N 33/68

(52) U.S. Cl. CPC G01N 33/56983; C12N 1/20; C12N 15/115; C12N 15/70; G01N 33/6893; C12N 2310/16; C12R 2001/19; G01N 2333/165

(57) ABSTRACT

The present invention relates to development of novel ssDNA aptamers comprising the oligonucleotide sequence that detect receptor-binding domain (RBD) of Severe Acute Respiratory Syndrome Corona virus (SARS-nCOV-2) infection. The ssDNA aptamers of the present invention may be used in lateral flow assays, electrochemical sensors and calorimetric sensors using gold nanoparticles. The present invention also relates to an analytical composition comprising said ssDNA aptamers. Further, the present invention also relates to a diagnostic kit for detecting RBD of SARS-nCoV-2.

### FIELD OF THE INVENTION

The present invention relates to development of novel ssDNA aptamers comprising the oligonucleotide sequence that detect receptor-binding domain (RBD) of Severe Acute Respiratory Syndrome Corona virus (SARS-COV-2) infection. The ssDNA aptamers of the present invention may be used in lateral flow assays, electrochemical sensors and calorimetric sensors using gold nanoparticles. The present invention also relates to an analytical composition comprising said ssDNA aptamers.

### BACKGROUND OF THE INVENTION

Viral infections can cause severe diseases in humans and remain one of the biggest challenges for human health. A near-perfect detection of viral infection is crucial for clinical therapy. Severe Acute Respiratory Syndrome Corona virus disease (SARS-nCOV-2) emerged in 2019 and claimed millions of lives. In response to nCOV-2 spread, rapid detection is crucial for estimating the severity of the disease and treating patients. Currently, there are several RT-PCR-based diagnostic kits available for SARS-nCOV-2 detection, which are time-consuming, expensive, and require advanced equipment facilities and trained personnel. The cost of RT-PCR diagnosis and insufficient test kits may prevent checking for viral transmission.

There has been an increasing interest in identifying and applying nucleic acid oligonucleotides, commonly known as aptamers, for detecting the viral proteins from infected patients. Thus, there is a need to develop an accurate, sensitive, cost-effective molecular method for the early detection of COVID-19 infection. Serology or antibody-based test kits are not recommended due to the presence of antibodies does not confirm whether a person is immune to COVID-19 or not. The aptamer is a next generation antibody mimic (chemical antibodies) diagnostic technology. Similar to antibodies, aptamers can bind specifically to viral proteins.

Notably, compared to antibodies, aptamers can bind to viral targets with high affinity and specificity, are more sensitive, and easier to synthesize. The DNA-based aptamers can be potentially used in diagnostics as well as in therapeutics of Coronavirus. Aptamers can be produced by Systematic Evolution of Ligands by Exponential Enrichment (SELEX) method.

Previously, aptamers specific for the viral proteins have been used as antiviral agents to rapidly detect viruses. Aptamers recognizing hemagglutinin (HA) proteins have been employed to detect various influenza virus strains, including H1N1, H3N2, H5N1, and H9N2. Similarly, for the medical diagnosis of Zika virus infection, NS1-binding aptamers can be applied. Moreover, aptamers may suppress viral infection by interfering with the enzymatic activities of virus-encoded enzymes.

As, the emergence of SARS COV-2 infection has caused a large global outbreak and is a major public health issue. An early and precise detection of viral infection is crucial for clinical therapy. In response to nCOV-2 spread, the rapid detection is very important for estimating the severity of the disease and treatment of patients. Currently, there are several RT-PCR based diagnostic kits, and other antigen test kits available for SARS-nCOV-2 detection, which are time-consuming, expensive, need advanced equipment facilities. Serology or antibody-based test kits are not recommended. Thus, there is a need to develop an alternative accurate, sensitive cost-effective molecular method for the early detection of nCOV-2 infection.

Coronavirus contains four structural proteins, such as spike(S), envelope (E), membrane (M), and nucleocapsid (N) proteins. The S protein has a receptor-binding domain (RBD) that mediates the first binding of the virus to a host receptor. The present invention has identified single-stranded DNA-aptamers that can specifically bind to the COVID-19 receptor-binding domain (RBD) of S protein. The present invention discloses that DNA aptamers can be used to develop a robust, sensitive, cost-effective, and efficient molecular diagnostic assay for detecting COVID-19 viral infection in the patient's sample.

Using an ACE2 competition-based aptamer selection strategy and a machine learning screening algorithm, Song et al. [Anal. Chem. 2020, 92, 14, 9895-9900] discovered SARS-nCOV-2 RBD-targeting aptamers with high binding affinity. The K_{d} values of the optimized CoV2-RBD-1C and CoV2-RBD-4C aptamers against RBD were 5.8 nM and 19.9 nM, respectively. Simulated interaction modeling, along with competitive with experiments, suggests that two aptamers may have partially identical binding sites at ACE2 on SARS-nCOV-2 RBD. These aptamers present an opportunity for generating new probes for recognition of SARS-nCOV-2 and could provide assistance in the diagnosis and treatment of SARS-nCOV-2 while providing a new tool for in-depth study of the mechanisms behind the coronavirus infection.

### OBJECTIVES OF THE INVENTION

The primary object of the present invention is to develop novel single standard DNA (ssDNA) oligonucleotide sequence (Aptamers). These novel aptamers produced from a synthetic library using Systematic Evolution of Ligands by Exponential Enrichment (SELEX) method for precise and rapid detection of s-protein of receptor-binding domain (RBD) of SARS-nCOV-2.

Secondly, the present invention is to provide a pharmaceutical composition comprising the aptamers for the SARS-nCoV-2.

Another object of the present invention is to provide a diagnostic kit for detection of SARS-nCOV-2 which comprises ssDNA aptamers.

### SUMMARY OF THE INVENTION

In an aspect, the present invention provides a synthetic oligonucleotide library of 5′-ATGCGGATCCCGCGC(N)₄₁GCGCGAAGCTTGCGC-3′, wherein the primer sequences 5′-ATGCGGATCCCGCGC-3′ (SEQ ID NO: 18) and 5′-GCGCGAAGCTTGCGC-3′ (SEQ ID NO: 19) anneal to the flanking regions of oligonucleotide library of ssDNA aptamers and are common in the development of all ssDNA aptamers of the present invention, wherein (N)₄₁ represents 41 nucleotides with equimolar A, G, C, and T incorporation at each position.

In an aspect, the present invention provides ssDNA aptamers for binding to RBD antigen of SARS-nCOV-2 selected from the group consisting of: an oligonucleotide sequence having at least 70% sequence identity (e.g., 75% identity, 80% identity, 85% identity, 90% identity, 95% identity, 98% identity, 99% identity, or 100% identity) with SEQ ID NOs: 1-9.

In an aspect, the present invention provides ssDNA aptamers for binding to RBD antigen of SARS-nCOV-2 selected from the group consisting of: an oligonucleotide sequence selected from anyone of SEQ ID NOs: 10-14.

In an aspect, the present invention provides ssDNA aptamers for binding to RBD antigen of SARS-nCOV-2 selected from the group consisting of: variants of oligonucleotide sequence selected from anyone of SEQ ID NOs: 10-14.

In another aspect, the present invention provides a process of expressing the ssDNA aptamers comprising the SEQ ID NOs: 1-14, said process comprising the steps of:
a. providing isolated oligonucleotide sequences of the ssDNA aptamers comprising the SEQ ID NOs: 1-14;
b. cloning said nucleotide sequences in an expression vector construct comprising pGEMT vector;
c. transforming the expression vector construct from step b) to E. coli DH5a cells; and
d. growing the transformed E. coli DH5a cells in culture media and inducing the expression of ssDNA aptamers.

In another aspect, the present invention provides a method for detecting RBD antigen of SARS-nCOV-2, comprising: contacting a sample containing RBD antigen of SARS-nCOV-2 with the labelled ssDNA aptamers of SEQ ID NOs: 1-14 of the present invention to form a RBD antigen-aptamer complex; measuring a signal from the E. coli-aptamer complex; and identifying a presence or concentration of SARS-nCOV-2 within the sample based on the measured signal.

In another aspect, the present invention provides a lateral flow device (LFD), an electrochemical sensor and calorimetric sensor for the rapid detection of RBD antigen of SARS-nCOV-2 comprising the ssDNA aptamers of SEQ ID NO:1-14.

In another aspect, the present invention provides a lateral flow device (LFD), an electrochemical sensor and calorimetric sensor for the rapid detection of RBD antigen of SARS-nCOV-2 comprising the ssDNA aptamers of SEQ ID NOs: 3, 6, 8, 10, 11, 12, 13, 14 and combinations thereof.

In yet another aspect, the present invention provides an analytical composition comprising one or more of the ssDNA aptamers of SEQ ID NOs: 1-14 for the rapid detection of RBD antigen of SARS-nCOV-2.

In yet another aspect, the present invention provides an analytical composition for the rapid detection of RBD antigen of SARS-nCOV-2 comprising ssDNA aptamers SEQ ID NOs: 3, 6, 8, 10, 11, 12, 13, 14 and combinations thereof. In yet another aspect, the present invention provides a diagnostic kit comprising i) ssDNA aptamers of SEQ ID NOs: 3, 6, 8, 10, 11, 12, 13, 14 and combinations thereof; ii) a binding buffer comprising Tris/Cl, NaCl, MgCl₂, pH 8.0, and BSA.

### BRIEF DESCRIPTION OF THE DRAWINGS

FIG. 1 illustrates a schematic representation of Systematic Evolution of Ligands by Exponential Enrichment (SELEX) for the generation of specific aptamers against the recombinant RBD protein of SARS-nCOV-2.

FIG. 2 illustrates SDS-PAGE analysis of RBD proteins of SARS-nCOV-2, Lanes EL1, EL2, EL3, and EL4 are elutes 1, 2, 3 and 4 of RBD proteins. Lane M: Protein molecular weight marker.

FIG. 3 illustrates SDS-PAGE of western blot analysis of RBD proteins in different elution EL1 and EL2 were detected in presence of anti-His-tag antibodies. Lane 1: elution 1 (EL1), and Lane 2: elution 1 (EL2), Lane M: protein ladder.

FIG. 4 illustrates PCR amplification of SELEX product resolved onto 2.5% agarose gel electrophoresis. Lane 1: Ladder 100 bp, Lane 2: Both forward and reverse primers and lane 3: Eluted DNA pool has shown a specific amplicon at ~80 bp.

FIG. 5 illustrates Prediction of secondary structure of developed aptamers library against receptor-binding domain (RBD) of spike antigen: a) AptRBD1; b) AptRBD2; c) AptRBD3; d) AptRBD4; e) AptRBD5; f) AptRBD6; g) AptRBD7; h) AptRBD8; and i) AptRBD9.

FIG. 6 illustrates Validation of Aptamers using APTA-ELISA for specific detection of RBD Antigen.

FIG. 7 illustrates Quantitative detection of novel aptamers for specific detection of RBD antigen.

FIG. 8 illustrates Interaction of AptRBD3 (stick structure) with L and H chain (ribbon structure) of RBD (7e30) of SARS COV2.

FIG. 9 illustrates Interaction of AptRBD6 (stick structure) with L and H chain (ribbon structure) of RBD (7e30) of SARS COV2.

FIG. 10 illustrates Interaction of AptRBD8 (stick structure) with L and H chain (ribbon structure) of RBD (7e30) of SARS COV2.

FIG. 11 illustrates engineering of aptamer for specific detection of RBD antigen of SARS-nCoV-2. a) AptRBD 3.1 aptamer, and b) AptRBD 3.2 aptamer, c) AptRBD 6.1 aptamer, and d) AptRBD 8.1 aptamers

### DETAILED DESCRIPTION OF THE INVENTION

The following is a detailed description of embodiments of the disclosure. The embodiments are to clearly communicate the disclosure. However, the of detail offered is not intended to limit the anticipated variations of embodiments; to cover all modifications, equivalents, and alternatives fall within the spirit and scope of the present invention.

It should also be appreciated that the present invention can be implemented in numerous ways, including as a system, a method or a device. In this specification, these implementations, or any other form that the invention may take, may be referred to as processes. In general, the order of the steps of the disclosed processes may be altered within the scope of the invention.

Embodiments of the present invention provides a synthetic oligonucleotide library of 5′-ATGCGGATCCCGCGC(N)₄₁GCGCGAAGCTTGCGC-3′, wherein the primer sequences 5′-ATGCGGATCCCGCGC-3′ (SEQ ID NO: 18) and 5′-GCGCGAAGCTTGCGC-3′ (SEQ ID NO: 19) anneal to the flanking regions of oligonucleotide library of ssDNA aptamers and are common in the development of all ssDNA aptamers of the present invention, wherein (N)₄₁ represents 41 nucleotides with equimolar A, G, C, and T incorporation at each position.

In some embodiments, the selection of aptamers using ssDNA library for specific detection of the recombinant His-tagged RBD protein comprises the steps of
a) incubating the ssDNA aptamers and the recombinant His-tagged RBD protein with Ni-NTA Sepharose beads;
b) washing out the non-specific ssDNA sequences 1-5 times with binding buffer;
c) separating the bounded mixture of ssDNA-RBD protein complex from the Ni-NTA beads by adding elution buffer, wherein the ssDNA-RBD protein complexes of each round are purified by the chemical method;
d) repeating the above steps for 6-8 sequential selection rounds, and after each round, the DNA library is enriched by PCR reaction for further rounds of SELEX.

In an aspect, the present invention provides ssDNA aptamers for binding to RBD antigen of SARS-nCOV-2 selected from the group consisting of: an oligonucleotide sequence having at least 70% sequence identity (e.g., 75% identity, 80% identity, 85% identity, 90% identity, 95% identity, 98% identity, 99% identity, or 100% identity) with SEQ ID NOs: 1-9.

In an aspect, the present invention provides ssDNA aptamers for binding to RBD antigen of SARS-nCOV-2 selected from the group consisting of: variants of oligonucleotide sequence having at least 70% sequence identity (e.g., 75% identity, 80% identity, 85% identity, 90% identity, 95% identity, 98% identity, 99% identity, or 100% identity) with SEQ ID NOs: 1-9.

In an aspect, the present invention provides ssDNA aptamers for binding to RBD antigen of SARS-nCOV-2 selected from the group consisting of: an oligonucleotide sequence selected from anyone of SEQ ID NOs: 10-14.

In an aspect, the present invention provides ssDNA aptamers for binding to RBD antigen of SARS-nCOV-2 selected from the group consisting of: variants of oligonucleotide sequence selected from anyone of SEQ ID NOs: 10-14.

In another aspect, the present invention provides a recombinant expression vector comprising one or more transcriptional regulatory elements operably linked to an oligonucleotide sequence encoding the one or more of the ssDNA aptamers of the present invention. In some embodiments, the expression vector is pGEMT vector.

In another aspect, the present invention provides a process of expressing the ssDNA aptamers comprising the SEQ ID NOs: 1-14, said process comprising the steps of:
a. providing isolated oligonucleotide sequences of the ssDNA aptamers comprising the SEQ ID NOs: 1-14;
b. cloning said nucleotide sequences in an expression vector construct comprising pGEMT vector;
c. transforming the expression vector construct from step b) to E. coli DH5a cells; and
d. growing the transformed E. coli DH5a cells in culture media and inducing the expression of ssDNA aptamers.

In an embodiment, the developed aptamers can be PCR amplified to enrich and scale-up of the product. The developed aptamers can be used for the specific detection of RBD antigen was evaluated by developing aptamer-based enzyme linked immunosorbent assay (APTA-ELISA) in presence of streptavidin protein that is covalently conjugated to horseradish peroxidase (HRP). The oligonucleotide sequences (aptamers) may be biotinylated with biotin-labelled forward primers. The binding efficiencies can be seen in presence of 3,3,5,5′-tetramethylbenzidine (TMB) by changing color from blue to yellow.

In another embodiment, the present invention provides ssDNA aptamers of SEQ ID NOs: 1-14 that can be used in in vitro and in vivo quantification of RBD antigen of SARS-nCOV-2, isolation of RBD antigen of SARS-nCOV-2, purification of RBD antigen of SARS-nCOV-2, and any combinations thereof.

In another embodiment, the ssDNA aptamers of SEQ ID NOs: 1-14 have a detectable label attached thereto. In some embodiments the detectable label is an optical label, an electrochemical label, a radioisotope or a combination thereof. The label may be attached to a certain base or certain structure of an ssDNA aptamer, for example, a certain site of a hairpin-loop structure or a 3′ end or a 5′ end of an aptamer.

In an embodiment, the optical label is selected from but not limited to fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, Comarin, Cy2 (cyanine 2), Cy3, Cy3.5, Cy5, Cy5.5, Cy-chrome, phycoerythrin, PerCP (peridinine chlorophyl-a protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor, 7-amino-4-methylcomarin-3-acetate, BODIPY FL, BODIPY FL-Br 2, BODIPY 530/550, their conjugates and mixture thereof.

In an embodiment, the electrochemical label may be selected from any electrochemical labels known in the art.

In another embodiment, the present invention provides a method for detecting RBD antigen of SARS-nCOV-2, comprising: contacting a sample containing RBD antigen of SARS-nCOV-2 with the labelled ssDNA aptamers of SEQ ID NOs: 1-14 of the present invention to form a RBD antigen-aptamer complex; measuring a signal from the E. coli-aptamer complex; and identifying a presence or concentration of SARS-nCOV-2 within the sample based on the measured signal.

In another embodiment, the present invention provides ssDNA aptamers of SEQ ID NOs: 1-14 that improve sensitivity, accuracy, limit of quantitation, signal/noise ratio, or any combinations thereof of an analytical method for RBD antigen of SARS-nCOV-2 in a given sample.

In another embodiment, the present invention provides a lateral flow device (LFD), an electrochemical sensor and calorimetric sensor for the rapid detection of RBD antigen of SARS-nCOV-2 comprising the ssDNA aptamers of SEQ ID NO:1-14.

In another embodiment, the present invention provides a lateral flow device (LFD), an electrochemical sensor and calorimetric sensor for the rapid detection of RBD antigen of SARS-nCOV-2 comprising the ssDNA aptamers of SEQ ID NOs: 3, 6, 8, 10, 11, 12, 13, 14 and combinations thereof.

In yet another embodiment, the present invention provides an analytical composition comprising one or more of the ssDNA aptamers of SEQ ID NOs: 1-14 for the rapid detection of RBD antigen of SARS-nCOV-2.

In yet another embodiment, the present invention provides an analytical composition for the rapid detection of RBD antigen of SARS-nCOV-2 comprising ssDNA aptamers SEQ ID NOs: 3, 6, 8, 10, 11, 12, 13, 14 and combinations thereof.

In another embodiment, the present invention provides an analytical composition for determining the extent of SARS-nCOV-2 infection state, determining effectiveness of a drug or a clinical trial, assessing subjects' response to the therapy, or any combinations thereof, wherein the composition comprises: one or more ssDNA aptamers comprising SEQ ID NOs: 1-14 conjugated with one or more contrast agents, microbubbles, liposomes, paramagnetic liposomes, or any combinations thereof.

In yet another embodiment, the present invention provides a diagnostic kit comprising i) ssDNA aptamers of SEQ ID NOs: 3, 6, 8, 10, 11, 12, 13, 14 and combinations thereof; ii) a binding buffer comprising Tris/Cl, NaCl, MgCl₂, pH 8.0, and BSA.

In yet another embodiment, the present invention provides a pharmaceutical composition comprising the aptamers selected from the group of ssDNA aptamers of SEQ ID NOs: 3, 6, 8, or combinations thereof and one or more pharmaceutically acceptable excipients.

In an embodiment of the present invention, the ssDNA aptamers of SEQ ID NOs: 1-14 may be linked to any drug moiety known in the art. For example, the drug moiety is selected from the group consisting of small molecules, polymers, antibodies (monovalent, polyvalent, and the like), nucleic acids (DNA, RNA, cDNA, and the like), organic compounds, inorganic compounds, and the like.

In yet another embodiment, the present invention provides a method of inhibiting SARS-nCOV-2 infection in a subject comprising the step of administering to the subject a composition comprising one or more ssDNA aptamers having a nucleotide sequence selected from the group consisting of SEQ ID NO:1-14, wherein the nucleotide sequences specifically bind to RBD antigen of SARS-nCOV-2 thereby reducing or treating or managing SARS-nCOV-2 infection.

In an embodiment of the present invention, the subject includes mammals including human subjects.

### EXAMPLES

### Deposition and Procurement Details:

1. pET-15b expression vector (Novagen, Madison, WI) procured from CSIR Institute, Delhi, India.
2. The RBD sequence of SARS-nCOV-2 from Wuhan strain; GB accession ID: NC_045512.
3. Isopropyl-d-thiogalactopyranoside (IPTG)-Invitrogen company, India
4. LB medium: HiMedia company, India
5. Ampicillin: HiMedia company, India
6. Tris-HCl: Sigma Aldrich company, India
7. NaCl: HiMedia company, India
8. Imidazole: Sigma Aldrich company, India
9. PMSF: Phenyl methyl sulfonyl fluoride: Sigma Aldrich company, India
10. Probond™ Ni-chelating resins: Thermo Fisher Scientific company, India
11. Sodium dodecyl sulfate-polyacrylamide: Sigma Aldrich company, India
12. RNAse and DNAse: HiMedia company, India
13. Mercaptoethanol: Sigma Aldrich company, India
14. Glycerol: SRL company, India
15. Bromophenol blue stain: Qualigens company, India
16. Tris-glycine buffer: Sigma Aldrich company, India
17. Coomassie blue stain: HiMedia company, India
18. Nitrocellulose membranes: BioRad company, India
19. Skim milk powder: HiMedia company, India
20. Twin 20: HiMedia company, India
21. Anti-His-tag monoclonal IgG: Invitrogen company, India
22. 3, 3′-diaminobenzidine (DAB) Western blot substrates: Sigma Aldrich company, India
23. Urea: HiMedia company, India
24. Sodium Phosphate: HiMedia company, India
25. Lysozyme: HiMedia company, India
26. NTA Sepharose beads: Qiagen company, India
27. MgCl₂: HiMedia company, India
28. BSA: HiMedia company, India
29. Phenol/chloroform/isoamyl alcohol (PCI): HiMedia company, India
30. Forward primer 5′ATGCGGATCCCGCGC-3: Eurofins Pvt Ltd company, India
31. Reverse primer 5′GCGCAAGCTTCGCGC-3′: Eurofins Pvt Ltd company, India
32. dNTPs: HiMedia company, India
33. Taq DNA polymerase: HiMedia company, India
34. 2-exonuclease enzyme: NEB, Germany
35. 5′-phosphorylated reverse primer: Eurofins Pvt Ltd company, India
36. DNA loading dye: HiMedia company, India
37. Agarose gel: Invitrogen company, India
38. pGEMT vector: Promega company, India
39. LB medium: HiMedia company, India
40. H2SO4: HiMedia company, India
41. ssDNA library: Eurofins Pvt Ltd company, India
42. 5′-biotinylated primer: Eurofins Pvt Ltd company, India.

### Example 1: Cloning and Protein Expression of RBD Gene of SARS COV2

The SARS-nCOV-2 RBD protein gene cDNA was cloned into a bacterial expression vector pET-15b expression vector (Novagen, Madison, WI). The RBD sequence was of SARS-nCOV-2 (Wuhan strain; GB accession ID: NC_045512). This constructs then transformed into E. coli strain DH5 and checked for orientation and N-terminal His tag by sequencing. The pET15b-His-RBD expression clone construct was transformed into E. coli strain BL21 Star™ (DE3). Protein expression was induced by the addition of 5 mmol/l isopropyl-d-thiogalactopyranoside (IPTG) in a 1 lit LB medium with ampicillin as a resistant marker. Cells were harvested by centrifugation at 3000 rpm at 4° C. for 15 mins. The thick bacterial pellet was suspended in binding buffer (20 mM Tris-HCl pH 7.8, 500 mM NaCl, 5 mM Imidazole, and 1 mM PMSF), sonicated, and centrifuged at 12,000×g at 4° C. for 30 min. The clear supernatant was applied onto the affinity column with Probond™ Ni-chelating resins (Invitrogen™). The column was washed with gradient washing buffers (20 mM Tris-HCl pH 7.9, 500 mM NaCl, 5—, 20-, 40-, and 60-mM Imidazole). The purified RBD protein was eluted with elution buffer (20 mM Tris-HCl pH 7.9, 500 mM NaCl, 100 mM Imidazole) and concentrated by dialysis against polyethylene glycol 6000.

### Western Blot

Sodium dodecyl sulfate-polyacrylamide gel electrophoresis (SDS-PAGE) was used to check the in vitro expression of SARS COV-2 RBD protein. Briefly, 5 ml samples of culture were removed just before the addition of IPTG and after induction, and cells were collected by centrifugation at 6000 rpm for 5 min. The cell pellets were suspended in 500 μl of sterile molecular biology grade water, and the sample was freeze-thawed twice to facilitate the disruption of the cells. 100 μl cell suspensions were treated with RNAse and DNAse (both at 40 μg/ml) to digest nucleic acids. After mixing with SDS-PAGE sample buffer containing SDS and—mercaptoethanol, Glycerol, Bromophenol blue was added. Protein denaturation and reduction were facilitated by heating samples at 95° C. for 5 minutes. Proteins were detected using SDS-polyacrylamide gels with Tris-glycine running buffer and Coomassie blue staining. Proteins separated by SDS-PAGE were transferred onto nitrocellulose membranes at 40 V (100 mA) for 2 h. Membranes were then incubated with 30 ml of PBS buffer (pH 7.4), containing 5% nonfat milk powder and 0.1% Twin 20 for 1 h at room temperature. The expressed proteins were probed with anti-His-tag monoclonal IgG raised in the rat and subsequently, anti-Rat IgG secondary polyclonal IgG antibodies (HRP labeled) raised in rabbits. After each antibody step, the blot was thoroughly washed thrice with 0.1% PBST on a rocker. The membranes were finally washed with TPBS for 10 min before the 3, 3′-diaminobenzidine (DAB) Western blot substrates were applied (Sigma Aldrich, India).

### Example 2: Expression and Purification of SARS-nCoV-2 RBD Protein

The present invention transformed the Escherichia coli BL21 cells with SARS-COV RBD protein expression vector and the cells were grown at 37° C. in LB broth. A 0.5 mM isopropyl-β-thiogalactopyranoside (IPTG) is added to induce the RBD protein expression and the cells were further incubated. To purify the expressed RBD protein, the cells were harvested by centrifugation (8000 rpm for 10 min) and re-suspended into the binding buffer (8 M Urea, 20 mM Sodium Phosphate and 500 mM Sodium Chloride, pH 7.8) and lysed by sonication followed by lysozyme treatment. The cell lysate was then centrifuged at 1000 rpm for 10 minutes. The supernatant was subjected to Nickel-NTA-bead chromatography for purification of RBD protein. In brief, the cell lysate was incubated with activated Ni-NTA beads for 1 hour with intermediate shaking. The unbound proteins were eluted by wash buffer (8 M Urea, 20 mM Sodium Phosphate and 500 mM Sodium Chloride, pH 6), and flow-through fractions containing the RBD protein were collected by washing the column with elution buffer (25 mM imidazole in binding buffer). The RBD protein was further confirmed by 12% SDS-PAGE and Western blot.

### Example 3: Systematic Evolution of Ligands by Exponential Enrichment (SELEX)

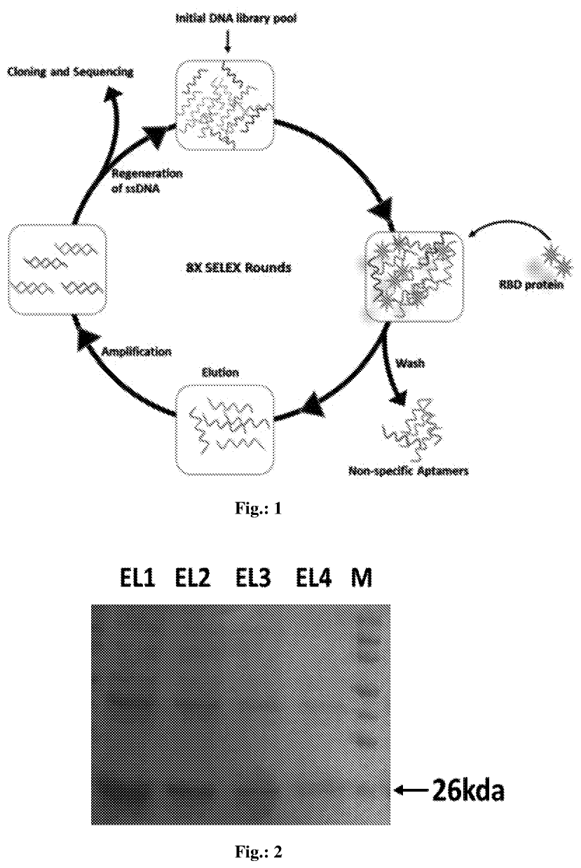

The selection of DNA aptamers specific to the recombinant RBD protein was performed as described previously. In brief, purified 5 μg of His-tagged RBD protein was incubated with 100 μL Ni-NTA Sepharose beads in the vertical column and washed once with binding buffer (50 mM Tris/Cl, pH 8.0, 150 mM NaCl, 1.5 mM MgCl₂, and 1% (w/v) BSA). The ssDNA library (5 μg/mL) was heated at 90° C. for 10 min, then immediately cooled on ice for 10 minutes, and then was incubated with the Ni beads-RBD protein complex for 30 min at room temperature with occasional shaking. The non-specific DNA sequences were washed five times with 500 μL of binding buffer and discarded. The mixture was further incubated for 30 minutes at room temperature. Finally, the RBD protein complexed with aptamers was separated from the Ni-NTA beads by adding 100 μL of elution buffer (binding buffer with 0.4 M imidazole) (FIG. 1).

The ssDNA-protein complexes of each round were purified by the chemical method using phenol/chloroform/isoamyl alcohol (PCI) treatment and then precipitation with 70% ethanol. Eight sequential selection rounds were repeated using the same procedure, and after each round, the DNA library was enriched by PCR reaction for further rounds of SELEX.

### Polymerase Chain Reaction

The DNA library consisted of two primer regions at the start and end of a 40-bases random region (5′-ATGCGGATCCCGCGC-N41-GCGCAAGCTTCGCGC-3′), where N41 relates to 41 nucleotides with equimolar A, G, C, and T incorporation at each position. All PCR reactions were carried out in a volume of 100 μL with the forward primer (5′ATGCGGATCCCGCGC-3), and reverse primer (5′GCGCAAGCTTCGCGC-3′). The PCR mixture (100 μL) contained 0.2 mM dNTPs, 0.5 μM each primer, 100 ng template and 2.5 U Taq DNA polymerase. The mixture was thermally cycled 35 times through 95° C. for 4 min, 94° C. for the 30 sec, and 60° C. for 45 sec, which was followed by a 1 min extension step at 72° C.

ssDNA Generation

Purified dsDNA was incubated with 25 U 2-exonuclease enzyme (NEB, Germany) in a total reaction volume of 50 μL at 37° C. for 3 h after each PCR cycle using the 5′-phosphorylated reverse primer. The reaction was then terminated by adding 10 μl of DNA loading dye. The products of the digested strand were analyzed by electrophoresis in a 2% agarose gel.

### Example 4: Cloning and Sequencing of DNA Aptamer

After the 8^{th} round of SELEX, ssDNA was amplified by PCR, and subsequently an A-tailing reaction was performed on the purified PCR product. A ligation reaction of the insert and pGEMT vector (Promega) was put for overnight incubation as per the manufacturer's instruction. The next day, 200 μl of competent cells was added into the ligation reaction tube and the tube was incubated on ice for 30 minutes. Later on, a heat-shock treatment was given to the cells for 90 seconds in the water at 42° C. Immediately the tubes were kept on ice for 3 minutes. Then 1 ml of LB medium was added to the tube and incubated in the incubator-shaker for an hour at 37° C. at 180 rpm. After the incubation, cells were spread on LB plates containing 100 μL of 20 mg/mL X-gal, 100 μL of 100 mM IPTG and 25 μL of 100 mg/mL Ampicillin. Plates were incubated overnight at 37° C. and examined for blue-white colonies.

### Enzyme-Linked-Aptamer Assay (ELAA)

An ELAA is a modified version of ELISA (Enzyme-Linked Immunosorbent Assay) for detecting binding of biotinylated aptamer with the target protein. In ELAA, aptamers are used as a substitution for primary antibodies. The purified RBD protein was loaded onto 96 well plates and incubated overnight at 4° C. The next day, plate was washed three times with PBS and blocked with 5% BSA in PBST at room temperature for 1 h. The plate was washed 2 times with washing buffer and then incubated with 5′-biotinylated ssDNA aptamer in PBST for 1 h. After 4 washes, the plate was incubated with streptavidin for 1 h and plates were observed for color development. The reaction was stopped by adding 1N H₂SO₄.

### Example 5: Expression of Recombinant RBD Protein

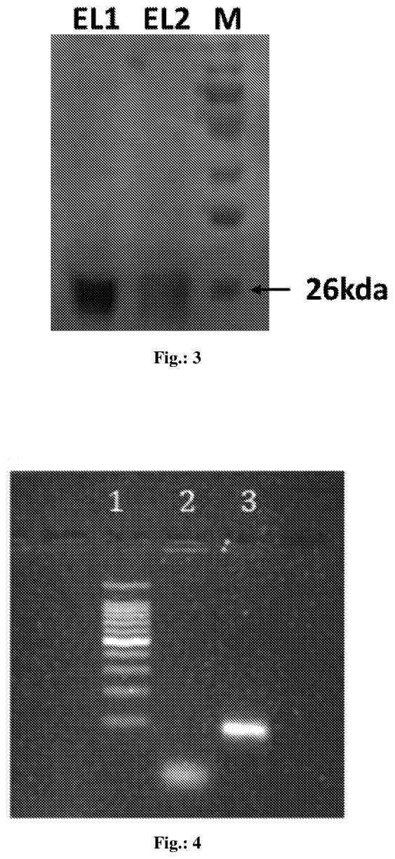

The present invention cloned the RBD of the S protein into the pET15b vector and transformed it into the E. coli strain BL21 (DE3). Protein expression was induced in LB medium by the addition of 5 mmol/L (IPTG) in LB medium with ampicillin. Inclusion body purification with chaotropic chemicals was used to purify the expressed protein. Standard assay procedures were used to quantify the purified protein. The molecular weight of the protein, 26 kDa was confirmed using 12% SDS-PAGE (FIG. 2). Further, western blots analysis performed using His-tag-specific antibodies which shown a protein blot at 26 kDa, confirming the RBD of SARS-nCOV-2 (FIG. 3).

### Example 6: Selection of ssDNA Aptamers Against RBD Protein

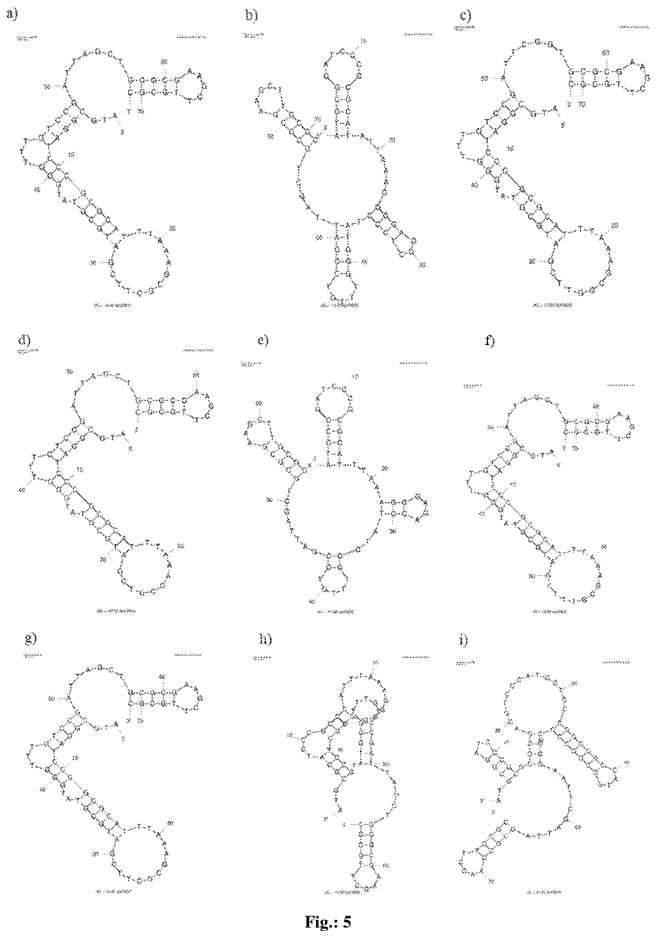

The random ssDNA library was screened with the recombinant RBD protein by the SELEX procedure. In this process, the RBD protein was immobilized onto Ni-NTA beads and then ssDNA library was added. The process eluted the DNA-protein complex was purified. The obtained oligonucleotide pool was confirmed by PCR. A single amplicon at 80 bp confirms the positive PCR was observed when resolved onto 2.5% agarose electrophoresis (FIG. 4).

In this study, a total of eight rounds of SELEX were performed against the target antigen. Furthermore, a total of six rounds with the target antigen and negative SELEX with the E. coli protein. After five rounds of SELEX, stringent conditions were provided to enhance the sensitivity of the aptamers. The PCR product was enriched and purified to remove the other salts from the reaction mixture and was used for the next round of SELEX to enhance the specificity. As the SELEX method requires ssDNA, the PCR product was incubated with λ-exonuclease to generate ssDNA from the dsDNA pool.

### Example 7: Cloning and Sequencing of the Aptamers

After eight successful rounds of SELEX, the purified DNA pool was amplified by PCR and cloned into a linearized pGEM T Easy vector to transform competent E. coli DH5a. The cells were plated onto Luria agar and incubated overnight. The resultant positive colonies were screened for colony PCR. The positive colony plasmid was extracted and enriched for the plasmid DNA and confirmed the insert DNA by aptamer-specific and vector-specific primers. The recombinant plasmids were sequenced with aptamer-specific primers. A total of nine aptamers were obtained, which showed diverse novel sequences (Table 1).

TABLE 1
Aptamers for precise detection of SARS−nCoV−2 Receptor Binding Protein

| | SEQ | | | | ΔS | |
|---|---|---|---|---|---|---|
| | ID | | ΔG | ΔH | cal/ | Tₘ |
| S. No. | NO: | Aptamer Sequence | kcal/mol | kcal/mol | (K · mol) | (° C.) |
| AptRBD1 | 1 | ATGCGGATCCCGCGCAT | −12.82 | −148.40 | −437.1 | 66.3 |
| | | TTAAAGCGCTTCGATGC | | | | |
| | | GTATGGGTTTGTCCGATT | | | | |
| | | AGCTGCGCGAAGCTTGC | | | | |
| | | GC | | | | |
| AptRBD2 | 2 | ATGCGGATCCCGCGCAT | −11.83 | −171.70 | −515.4 | 59.9 |
| | | ATTAAAGGGGAGGCTCC | | | | |
| | | CTATGGGTTTGTCCGATT | | | | |
| | | AGCTGCGCGAAGCTTGC | | | | |
| | | GC | | | | |
| AptRBD3 | 3 | ATGCGGATCCCGCGCAT | −12.82 | −148.40 | −437.1 | 66.3 |
| | | TTAAAGCGGTTCGATGC | | | | |
| | | GTATGGGTTTGTCCGATT | | | | |
| | | CGGTGCGCGAAGCTTGC | | | | |
| | | GC | | | | |
| AptRBD4 | 4 | ATGCGGATCCCGCGCAT | −13.02 | −148.40 | −436.4 | 66.8 |
| | | TTAAAGCGTCGATGCGT | | | | |
| | | ATGGGTTTGTCCGATTA | | | | |
| | | GCTGCGCGAAGCTTGCG | | | | |
| | | C | | | | |
| AptRBD5 | 5 | ATGCGGATCCCGCGCAT | −11.38 | −147.80 | −439.8 | 62.8 |
| | | TTAAAGGGAGACCTATG | | | | |
| | | GGTTTGTCCGATTAGCT | | | | |
| | | GCGCGAAGCTTGCGC | | | | |
| AptRBD6 | 6 | ATGCGGATCCCGCGCAT | −12.82 | −148.40 | −437.1 | 66.3 |
| | | TTAAAGCGCTTCGATGC | | | | |
| | | GTATGGGTTTGTCCGATT | | | | |
| | | AGCTGCGCGAAGCTTGC | | | | |
| | | GC | | | | |
| AptRBD7 | 7 | ATGCGGATCCCGCGCAT | −12.82 | −148.40 | −437.1 | 66.3 |
| | | TTAAAGCGCTTCGATGC | | | | |
| | | GTATGGGTTTGTCCGATT | | | | |
| | | AGCTGCGCGAAGCTTGC | | | | |
| | | GC | | | | |
| AptRBD8 | 8 | ATGCGGATCCCGCGCAT | −11.90 | −143.40 | −423.9 | 65 |
| | | TTAAAGGGGGCGCTCCG | | | | |
| | | TATGGGTTTGTCCGATTA | | | | |
| | | GCTGCGCGAAGCTTGCG | | | | |
| | | C | | | | |
| AptRBD9 | 9 | ATGCGGATCCCGCGCCC | −21.55 | −201.30 | −579.5 | 74.1 |
| | | GACGTCGCATGCTCCCG | | | | |
| | | GCCGCCATGGCGGCCGC | | | | |
| | | GGGAATTCGATTAGCGC | | | | |
| | | GAAGCTTGCGC | | | | |

Thermodynamics of Folding: ΔG=ΔH−TΔS
Ionic conditions: [Na⁺]=1.0 M, [Mg⁺⁺]=0.0 M.

### Prediction of the Secondary Structure of the Aptamer Sequences

After cloning and sequencing, the candidate's secondary structure was predicted and their Thermodynamics of Folding: ΔG=ΔH−TΔS was calculated at ionic conditions: [Na⁺]=1.0 M, [Mg⁺⁺]=0.0 M. The predicted secondary structures of the aptamers are shown in FIG. 5.

### Aptamer (APT) Based ELISA (APTA-ELISA)

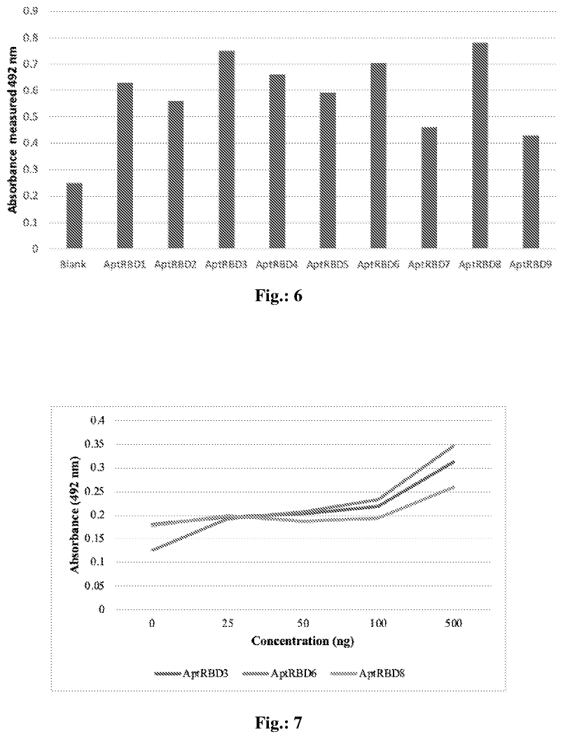

The present invention performed an enzyme-linked aptamer assay to assess the binding affinities of the generated aptamers, which specifically recognize the RBD antigen. In brief, aptamer sequences were biotinylated using a 5′-biotinylated primer. Furthermore, all these aptamers showed good sensitivity. The absorbance of each aptamer sequence was varied with the arrangement. Among nine novel aptamers, AptRBD3, AptRBD6 and AptRBD8 have shown good absorbance of 0.75, 0.70 and 0.75, respectively (FIG. 6).

The promising sequences also aligned using CLUSTAL O (1.2.4) multiple sequence alignment. All the sequences were novel and recognise precisely the RBD antigen of the SARS-COV2.

### CLUSTAL O (1.2.4) Multiple Sequence Alignment

| | | |
|---|---|---|
| AptRBD6 | ATGCGGATCCCGCGCATTTA-AAGGGGGCGCTCCGTATGGGTTTGTCCGATTAGCTGCGC | 59 |
| AptRBD3 | ATGCGGATCCCGCGCATTTAAAGCGGTTCGATGCGTATGGGTTTGTCCGATTCGGTGCGC | 60 |
| AptRBD8 | ATGCGGATCCCGCGCATTTAAAGCGTTTCGATGCGTATGGGTTTGTCCGATTAGCTGCGC | 60 |
| | ******************** * * ** * ******************* * ***** | |
| AptRBD6 | GAAGCTTGCGC | 70 |
| AptRBD3 | GAAGCTTGCGC | 71 |
| AptRBD8 | GAAGCTTGCGC | 71 |
| | *********** | |

These three (AptRBD3, AptRBD6 and AptRBD8) promising aptamers were further validated for their sensitivity. The listed three novel ssDNA aptamer sequences have shown sensitivity at 4-10 ng/ml of the RBD antigen. However, the absorbance increased with increasing protein concentration (FIG. 7). Aptamer AptRBD6 showed good sensitivity when compared with AptRBD3, and AptRBD8. In conclusion, aptamer AptRBD3 can be used to develop a lateral flow device for the rapid detection of SARS-COV2 infection in humans. It would be portable, low-cost, precise, and can detect antigens at even lower than 25 ng/μL of antigen. Such aptamers are also reported to be helpful in the neutralization of the SARS-COV2 antigen. However, novel developed aptamers were validated with the other aptamers published by Song et al. [Anal. Chem. 2020, 92, 14, 9895-9900] discovered SARS-nCOV-2 RBD-targeting aptamers with high binding affinity. In the present invention aptamers, namely AptRBD3, AptRBD6 and AptRBD8 have showed good sensitivity when compared with the Song et al. (2020).

Molecular Docking Approach to Check the Binding of Peptide with RBD of Covid 19

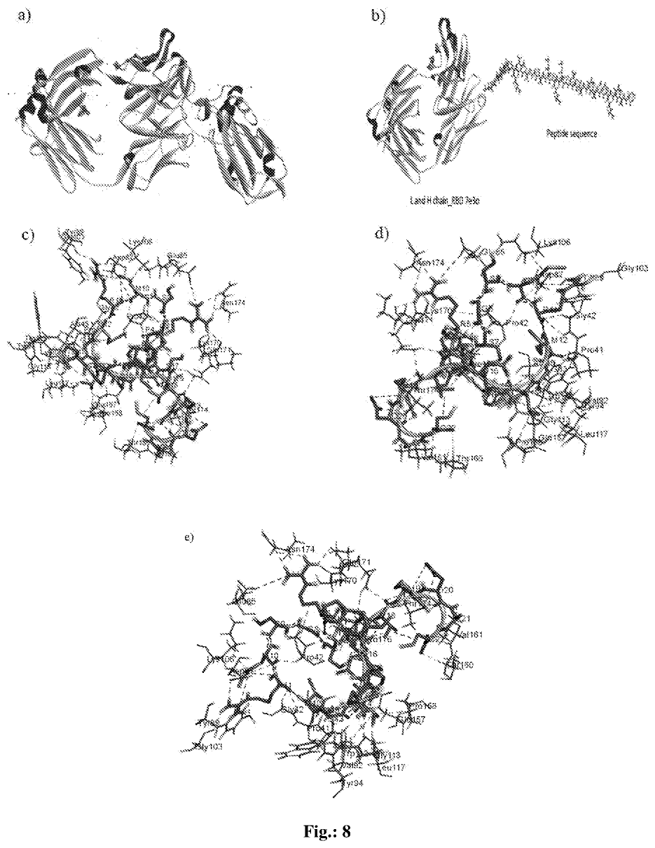

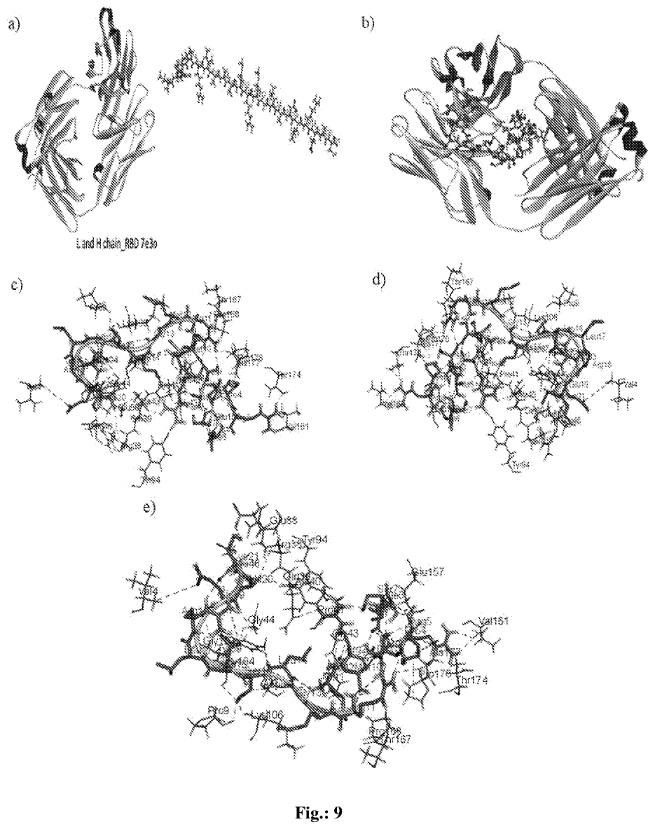

The interactions of RBD of Covid 19 with the selected putative aptamer (AptRBD6) was confirmed by using Auto dock Vina (5.1) molecular docking tool. The RBD of Covid 19 has 3 main chains as L, H, and R. Peptides mainly interact with L and H chain of the RBD protein. The binding energy of RBD and peptide 1 (MRIPRISGSMRMGLSDSVREAC) was-6.8 kcal/mol. AptRBD3 was interacted with Land H chain of the RBD protein by H-bonding and hydrophobic interactions (FIG. 8 a, and b). Amino acids Asn 174, Glu 85, Lys 106, Asp 87, GLY103, TYR89, GLY42, PRO41, VAL92, TYR 94, LEU117, PRO42, ALA45 TRP112, GLY113, GLU157, PRO 158, THR160, VAL161, THR174, GLN171, LYS170 were taken part in interaction (FIG. 8 c, d and e).

The interactions of RBD of Covid 19 with the selected putative aptamer (AptRBD6) was checked with Auto dock Vina. Peptides mainly interact with L and H chain of the RBD protein. The binding energy of RBD and peptide 2 (MRIPRISASMRMGLSDLREAC) was −6.3 kcal/mol. Peptide 2 also interacted with Land H chain of the RBD protein by H-bonding and hydrophobic interactions (FIG. 9 a, and b). Amino acids PRO9, LYS106, THR167, PRO168, PRO176, ALA177, THR174, VAL161, GLU157, PRO 42, PRO41, TYR94, ARG38, GLN39, ALA40, GLU88, GLU46, ASP87, GLY44, VAL4, GLY104 were taken part in interaction (FIG. 9 c, d and e).

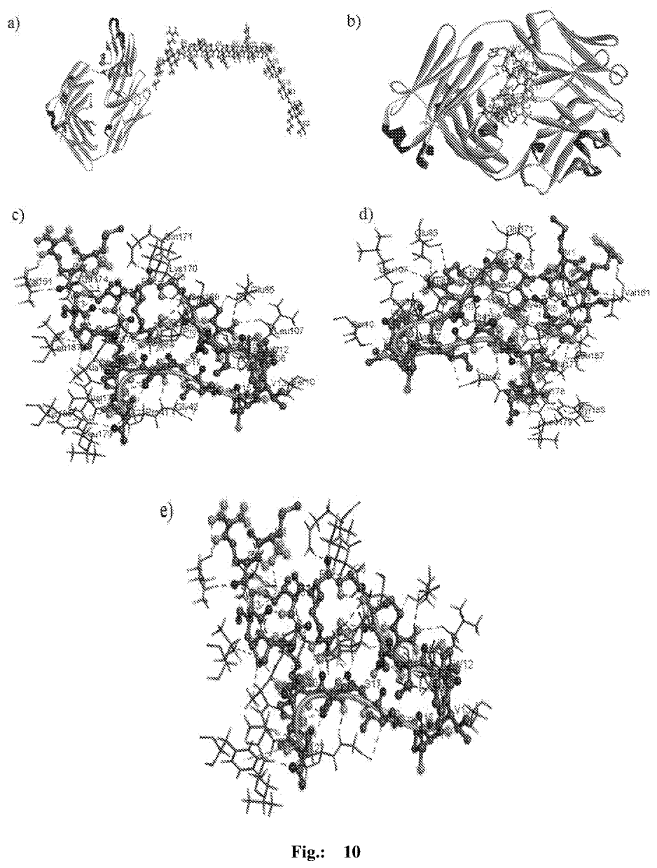

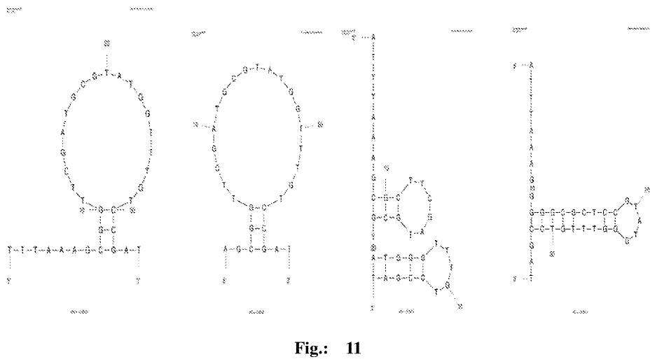

The interactions of RBD of Covid 19 with the selected putative peptide 3 was checked with Auto dock Vina. Peptides mainly interact with L and H chain of the RBD protein. The binding energy of RBD and peptide 3 (MRIPRIRGRSVWVCPISCAKLA) was-7.1 kcal/mol. AptRBD8 aptamer also interacted with Land H chain of the RBD protein by H-bonding and hydrophobic interactions (FIG. 10 a, and b). Amino acids GLN171, LYS170, SER169, GLU85, LEU107, ASP7, ALA117, SER10, GLY42, PRO41, LEU179, TYR185, VAL178, ALA177, LEU187, PRO176, GLY43, PRO42, PRO168, THR174, VAL161 were taken part in interaction (FIG. 10 c, d and e).

### Homology Modelling of Putative Aptamer Sequences to Obtain Peptide

The selected aptamer sequences were converted into the peptide sequence using the Discovery studio2017R2 software (Dassault Systemes BIOVIA) and converted into the PDB format. The aptamer AptRBD3-sequence ATGCGGATCCCGCGCA TTTAAAGCGGT TCGATG CGTATG GTTTGTCCGATTCGGTGCGCGAAGCTTGCGC had the MRIPRISGSMRMGLSDSVREAC (peptide 1, SEQ ID NO: 15) peptide sequence. Further, aptamer AptRBD6 sequence ATGCGGATCCCGCGC ATTTAAAGCGCTTCGATGCGTATGGGTTTGTCCGATTAGCTGCGCGAAGCTTGCGC aptamer sequence had MRIPRISASMRMGLSDLREAC (peptide 2, SEQ ID NO: 16) peptide sequence. Moreover, the aptamer AptRBD8 sequence ATGCGGATCCCGCGCATTTAAAGGGGGCGCTCCGTATGGGTTTGTCCGATTAGCTG CGCGAAGCTTGCGC had MRIPRIRGRSVWVCPISCAKLA (peptide 3, SEQ ID NO: 17) peptide sequence.

Molecular Docking Approach to Check the Binding of Peptide with RBD of Covid 19

Molecular docking software Autodock Vina 5.1 was used to study the interaction designed peptide with RBD of Covid 19. The crystal structure of RBD (RBD PDB id 7E30 Resolution 2.5 Å) was obtained from Research Collaboratory for Structural Bioinformatics PDB (RCBS PDB). The putative peptide sequences were converted to PDB format using Discovery studio2017R2. In the blind docking study, the whole peptide and RBD region was covered with a grid box set to 40 A°×40 A°×40 A° (x, y and z) having 1 A° spacing between grid points to remove the bias. The conformers generated were selected based on the lowest binding energy conformation of the final pose of ligand bound with the peptide. The interaction of RBD with, designed peptides were analyzed in detail with visualization tool Discovery studio2017R2 to find out the amino acid involved in interaction. Based on the molecular docking and APTA-ELIA results, AptRBD 3, AptRBD 6, and AptRBD 8 has exhibited good binding score against RBD of SARS-COV2. In the engineering of aptamers, the both primer sequences were removed, because these primers needed in PCR amplification, but actual prototype development, primers will be excluded. The core aptamer sequences were engineered to form or increase the loop size (FIG. 11). Aptamer AptRBD 3.1 showed bigger loops structure of the aptamers helps in more binding of RBD around it. However, the stalk is also play important in the attachment of the base of the prototypes. Hence, another five initial nucleotides were removed from the core part of the aptamers is designated as 3.2. Further, the engineered structures were further validated for their binding efficacious by APTA-ELISA. Interestingly, the absorbance of the engineered aptamer also could able to detect the RBD at 4 ng/mL increased to more than one-fold. In each aptamer sequence consists of both 15 forward and reverse primers sequences each. While, doing the ELISA the primers sequences also may influences the binding score or reactivity against to the RBD. However, primer sequences are excluded in any final prototype design of lateral flow device. It indicates the core central part is the actual aptamers in all the aptamer sequences. Hence, in the engineering of the aptamers, we have excluded the primers. In addition, we have sequentially deleted the single nucleotide to get very bulgy structure along with the three-nucleotides stalk. This kind of large looped structure aptamer can be used not only in the development of lateral flow device, but also electrochemical sensors. In electrochemical sensor, binding of the target-aptamer changes current due to alteration in distance between the redox tag and the electrode surface. This change in the electron transfer efficiency of redox tag to electrode can be detected. However, AptRBD 6, and AptRBD 8 aptamers could not form the single looped structure. Hence, AptRBD-3 and its engineered aptamer AptRBD 3.1, and AptRBD 3.2 can be used in the diagnosis of the RBD antigen of SARS-COV2. To the best of our knowledge, no engineered aptamers were reported to enhance the binding score or reactivity (Table 2).

TABLE 2

| | | SEQ ID | | LOD/ |
|---|---|---|---|---|
| Aptamer | Engineered Sequence 5′ to 3′ | NO: | Length | ng |
| AptRBD3 | ATGCGGATCCCGCGCATTTAAAGCGGTT | 3 | 70 | 10 |
| | CGATGCGTATGGTTTGTCCGATTCGGTGC | | | |
| | GCGAAGCTTGCGC | | | |
| AptRBD3.1 | ATTTAAAGCGGTTCGATGCGTATGGTTTGT | 10 | 40 | 4 |
| | CCGATTCGGT | | | |
| AptRBD3.2 | ATTTAAAGCGGTTCGATGCGTATGGTTTGT | 11 | 35 | 4 |
| | CCGAT | | | |
| AptRBD3.3 | AGCGGTTCGATG CGTATGGTTTGTCCGAT | 12 | 29 | 4 |
| AptRBD6 | ATGCGGATCCCGCGCATTTAAAGCGCTT | 6 | 71 | 7 |
| | CGATGCGTATGGGTTTGTCCGATTAGCTG | | | |
| | CGCGAAGCTTGCGC | | | |
| AptRBD6.1 | ATTTAAAGCGCTTCGATGCGTATGGGTTTG | 13 | 36 | 4 |
| | TCCGAT | | | |
| AptRBD8 | ATGCGGATCCCGCGCATTTAAAGGGGGC | 8 | 70 | 10 |
| | GCTCCGTATGGGTTTGTCCGATTAGCTGC | | | |
| | GCGAAGCTTGCGC | | | |
| AptRBD8.1 | ATTTAAAGGGGGCGCTCCGTATGGGTTTG | 14 | 40 | 8 |
| | TCCGATTAGCT | | | |

* Note:
Primers highlighted with bold and underlined fonts

### Advantages of the Invention

Aptamer is a next-generation antibody which mimics (chemical antibodies) diagnostic technology. As equal capacity to antibodies, aptamers can bind specifically to the target molecules and proteins with high affinity, are more sensitive and easier to synthesize. ssDNA aptamer(s) potentially used in diagnostics can be produced by several rounds of the SELEX (systematic evolution of ligands by exponential enrichment) method. These short nucleotide sequences can be easily produced by amplification PCR and chemically modified as per interest. An aptamer that detects specific antigen can develop diverse range of diagnostic tools. The aptamers proposed in this invention is capable of detecting SARS-nCOV-2 RBD protein and it can be developed as a lateral flow device (LFD) for rapid detection of SARS-nCOV-2 infection.

Detection of the Coronavirus by qRT-PCR and antigen (Ag) based test kits is the most popular method. Moreover, these methods are expensive, time-consuming, and have a high rate of false-positive results. To overcome the limitations of existing molecular diagnosis methods, the present invention has developed rapid, cheap, and robust single-strand DNA to diagnose the SARS-COV2 virus using single-stranded DNA (ssDNA) aptamer.

Aptamers are small ssDNA segments of 10-100 nucleotides that specifically bind to viral proteins.

However, our DNA aptamer sequences are unique to already reported sequences of aptamers for specific detection of the SARS-COV2 virus.

To identify single-stranded DNA-aptamers to develop a cost-effective and accurate molecular diagnostic kit that may be applied to detect COVID-19 viral infection.

1. A single stranded DNA (ssDNA) aptamer for binding to receptor-binding domain (RBD) of Severe Acute Respiratory Syndrome Corona virus (SARS-nCoV-2), said ssDNA aptamer comprising: an oligonucleotide selected from the group consisting of SEQ ID NOs: 1-14 and variants thereof.

2. The ssDNA aptamer as claimed in claim 1, comprising an oligonucleotide sequence having at least 80% similarity to SEQ ID NO: 3.

3. The ssDNA aptamer as claimed in claim 2, wherein the ssDNA aptamer translates to a peptide of SEQ ID NO: 15.

4. The ssDNA aptamer as claimed in claim 1, comprising an oligonucleotide sequence having at least 80% similarity to SEQ ID NO: 6.

5. The ssDNA aptamer as claimed in claim 4, wherein the ssDNA aptamer translates to a peptide of SEQ ID NO: 16.

6. The ssDNA aptamer as claimed in claim 1, comprising an oligonucleotide sequence having at least 80% similarity to SEQ ID NO: 8.

7. The ssDNA aptamer as claimed in claim 6, wherein the ssDNA aptamer translates to a peptide of SEQ ID NO: 17.

8. The ssDNA aptamer as claimed in claim 1, wherein the ssDNA aptamer has a detectable label attached thereto.

9. The ssDNA aptamer as claimed in claim 8, wherein the detectable label is an optical label, an electrochemical label, a radioisotope or a combination thereof.

10. A process of expressing the ssDNA aptamers of claim 1, said process comprising the steps of:
a) providing isolated oligonucleotide sequences of the ssDNA aptamers comprising the SEQ ID NOs: 1-14;
b) cloning said oligonucleotide sequences in an expression vector construct comprising pGEMT vector;
c) transforming the expression vector construct from step b) to E. coli DH5a cells; and
d) growing the transformed E. coli DH5a cells in culture media and inducing the expression of the ssDNA aptamers.

11. An analytical composition comprising at least one of the ssDNA aptamers as claimed in claim 1 and a binding buffer comprising Tris/Cl, NaCl, MgCl₂, pH 8.0 and BSA.

12. A method for detecting RBD antigen of SARS-nCOV-2, said method comprising:
a) contacting a sample containing RBD antigen of SARS-nCOV-2 with the labelled ssDNA aptamer as claimed in claim 9 to form a RBD antigen-aptamer complex;
b) measuring a signal from the E. coli aptamer RBD antigen-aptamer complex; and
c) identifying a presence or concentration of SARS-nCOV-2 within the sample based on the measured signal.

13. A SARS-nCOV-2 detection sensor comprising at least one of the ssDNA aptamers as claimed in claim 1, wherein the detection sensor is selected from a lateral flow device (LFD), an electrochemical sensor, and a calorimetric sensor.

14. A kit for detecting RBD of SARS-nCOV-2, said kit comprising:
a) at least one of the ssDNA aptamers as claimed claim 1; and
b) a binding buffer comprising Tris/Cl, NaCl, MgCl₂, pH 8.0, and BSA.
